(19) United States
(12) Patent Application Publication
(10) Pub. No.: US 2018/0064573 A1
(43) Pub. Date: Mar. 8, 2018

(54) Snore Reduction Device and Method

(71) Applicant: Snore Block, LLC, Tiverton, RI (US)

(72) Inventors: Curtis J. Mello, Tiverton, RI (US); Patricia Mello, Tiverton, RI (US)

(21) Appl. No.: 15/811,574

(22) Filed: Nov. 13, 2017

Related U.S. Application Data
(63) Continuation-in-part of application No. PCT/US2017/023105, filed on Mar. 18, 2017.
(60) Provisional application No. 62/313,605, filed on Mar. 25, 2016.

(51) Int. Cl. A61F 5/56; A47G 9/10

(52) U.S. Cl. CPC A61F 5/56; A47G 9/1045; A47G 9/109

(57) ABSTRACT

A sleep assist device suitable for placement against at least the shoulder blade of a user, and methods of making and using same. The device has a generally quadrilateral cross-section with a top surface, a bottom surface, a rear surface, an inclined front surface, and a bendable, conformable retention tab extendable along the bed away from the bottom surface. Preferably, the front surface is at an angle ranging from thirty five degrees to eighty degrees, and more preferably forty degrees to sixty five degrees, relative to the bottom surface.

### CROSS-REFERENCE TO RELATED APPLICATION

This application is a continuation-in-part application of International Patent Application No. PCT/US2017/023105 filed 18 Mar. 2017 which claims priority to U.S. Provisional Application No. 62/313,605 filed 25 Mar. 2016. The entire contents of each of the above-mentioned applications are incorporated herein by reference.

### FIELD OF THE INVENTION

This invention relates to techniques to reduce snoring while sleeping and more particularly to sleep-assist devices that encourage users to turn on their sides while in bed.

### BACKGROUND OF THE INVENTION

Snoring is a common problem that arises during sleep. Snoring includes shortness of breath while sleeping and often results in load noises that disturb partners in bed. Sleep deprivation for snorers and their partners is a common result. Physiological, psychological and social damages may arise from snoring. Other sleep disorders include sleep apnea, insomnia, and breathing difficulty.

It is known that people who sleep on their back may experience the tongue moving toward the back of the mouth which restricts air passageways. Types of orthopaedic pillows have been developed to correct body positioning, including cervical or contour pillows having a curved design intended to match head, neck and shoulder contours of sleepers. Wedge pillows are triangle-shaped pillows that provide a slope to place the body in a diagonal position, primarily to reduce symptoms of GERD (gastroesophageal reflux disease) and acid reflux while sleeping.

Other treatments for snoring include dental appliances, PAP (positive airway pressure) equipment, surgery, and/or medication. These treatments have different costs and may have side effects ranging from discomfort to medical complications.

It is therefore desirable to have a relatively inexpensive, non-invasive solution to sleep disorders including snoring.

### SUMMARY OF THE INVENTION

An object of the present invention is to provide an improved sleep-assist device which encourages users to turn on their sides in bed and decrease the likelihood of snoring.

Another object of the present invention is to provide such a device which is relatively easy to manufacture and which is relatively inexpensive.

This invention features novel use of a sleep assist device which has a generally quadrilateral cross-section with a top surface, a bottom surface which serves as a base, a rear surface and an inclined front surface. The device further includes a bendable retention tab extending at least six inches from the bottom surface. Preferably, the front surface is at an effective angle ranging from thirty five degrees to eighty degrees, and more preferably forty degrees to sixty five degrees, relative to the bottom surface.

In some embodiments, the sleep assist device is substantially trapezoidal in cross-section, with the top and bottom surfaces substantially parallel to each other. In certain embodiments, the front surface is at an angle ranging from forty five degrees to sixty degrees relative to the bottom surface.

This invention further features a method to reduce the manifestation of at least one sleep disorder by selecting a sleep assist device which has a generally quadrilateral cross-section with a top surface, a bottom surface, a rear surface, an inclined front surface, and a bendable retention tab extending at least six inches from the bottom surface. The method further includes placing the sleep assist device on a bed with the bottom surface contacting the bed with the retention tab extending along the bed away from the bottom surface, and placing at least a portion of at least one shoulder blade of a user on the inclined front surface of the sleep assist device and another portion of the user on the retention tab. This invention also features manufacturing such a sleep assist device.

### BRIEF DESCRIPTION OF THE DRAWINGS

In what follows, preferred embodiments of the invention are explained in more detail with reference to the drawings, in which:

FIG. 1 is a schematic perspective view of a novel sleep assist device;

FIG. 2 is a schematic perspective view of another novel sleep assist device;

FIG. 3 is a schematic top view of the device of FIG. 2 placed according to the present invention against the back of a user in bed;

FIG. 4 is a schematic side view of yet another sleep assist device according to the present invention illustrating “generally quadrilateral”;

FIG. 5 is a schematic perspective view of an improved sleep assist device according to the present invention with a bendable retention tab deployed on a bed; and

FIGS. 6A and 6B are schematic perspective views of the device and bed of FIG. 5 as utilized by a user during sleep.

### DETAILED DESCRIPTION OF THE PRESENTLY PREFERRED EMBODIMENTS

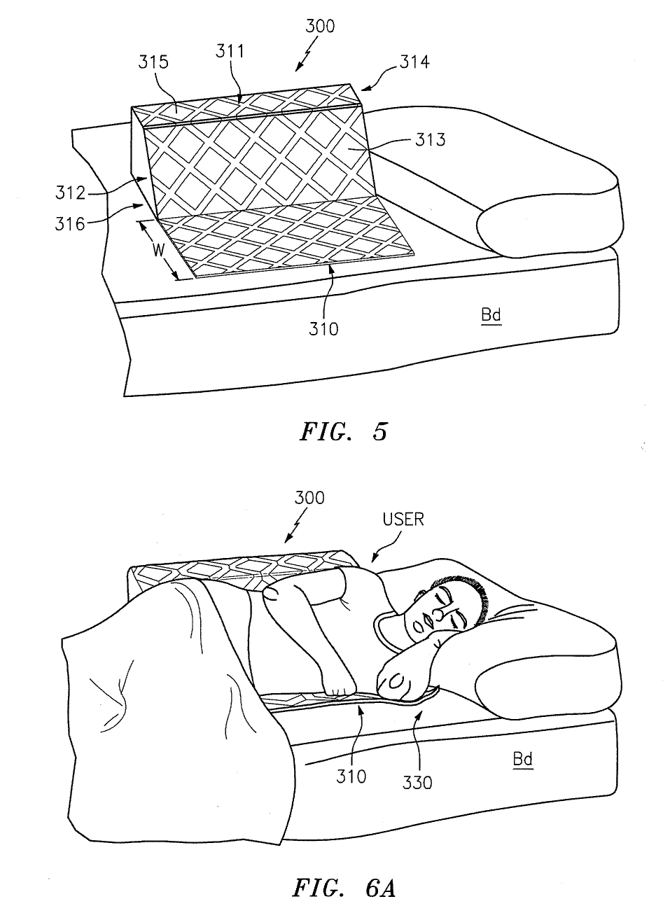

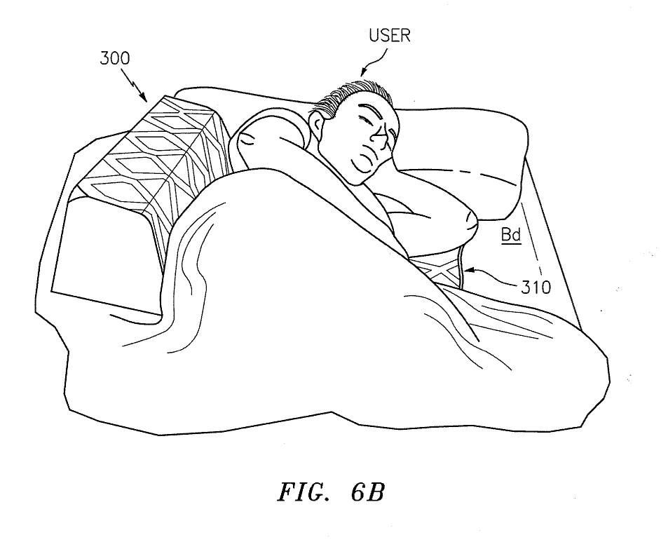

This invention may be accomplished by methods of making and using a novel sleep assist device to reduce the manifestation of at least one sleep disorder such as snoring. The sleep assist device has a generally quadrilateral cross-section with a top surface, a bottom surface which serves as a base, a rear surface and an inclined front surface which preferably is at an effective angle ranging from thirty five degrees to eighty degrees relative to the bottom surface, more preferably between forty degrees to sixty five degrees. A bendable retention tab extending at least six inches from the bottom surface as described below in more detail in relation to FIGS. 5-6B.

The term “generally” as utilized herein refers to at least a majority of a recited feature, such as “generally quadrilateral” described below in relation to FIG. 4.

The term “effective” as utilized herein refers to the actual parameter experienced during use of a device according to the present invention, such as an “effective angle” of the inclined front surface after the device bears some of the weight of a user.

The term “substantially” as utilized herein encompasses deviations of up to ten percent, such as a top surface and a bottom being “substantially parallel” encompassing deviations up to ten degrees from each other.

Sleep assist device 10, FIG. 1, has a front inclined surface 12, a top surface 14, a bottom surface 16 which serves as a base, and a back surface 18. Sleep assist device 10 also has an end surface 20 which is visible in FIG. 1, and an opposite end surface 22. In one construction, front surface 12 and top surface 14 form an angle α₁ that is approximately 45 degrees, front surface 12 and bottom surface 16 form an angle θ₁ that is approximately 135 degrees, and angles β₁ and C₁ are both right angles.

In the construction illustrated in FIG. 1, sleep assist device 10 has an upper depth D₁₁ of four inches, a lower depth D₁₂ of 10 inches, a height H₁ of six inches, and lengths L₁₁ and L₁₂ of twenty four inches.

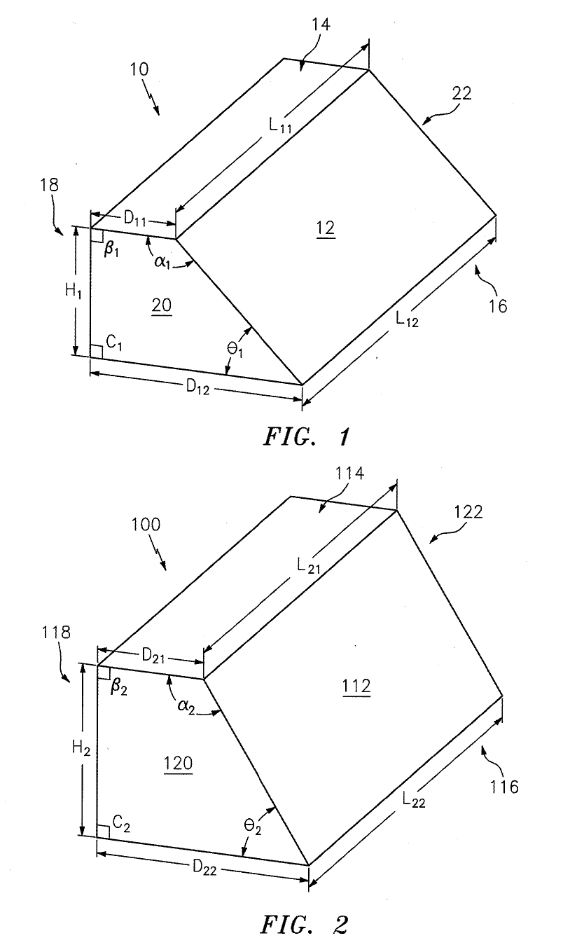

Sleep assist device 100, FIG. 2, has a front inclined surface 112, a top surface 114, a bottom surface 116, and a back surface 118. Sleep assist device 100 also has an end surface 120 which is visible in FIG. 2, and an opposite end surface 122. In one construction, front surface 112 and top surface 114 form an angle α₂ that is approximately 60 degrees, front surface 112 and bottom surface 116 form an angle θ₂ that is approximately 120 degrees, and angles β₂ and C₂ are both right angles.

In the construction illustrated in FIG. 2, sleep assist device 100 has an upper depth D₂₁ of four to five inches, a lower depth D₂₂ of about ten inches, a height H₂ of eight to nine inches, and lengths L₂₁ and L₂₂ of at least twelve inches, preferably twenty to thirty inches, and more preferably about twenty four inches. The height and length dimensions can be selected to match anticipated anatomical dimensions of the user as described in more detail below. The bottom surface 116 has a sufficient lower depth D₂₂ to provide stability during use.

Both sleep assist devices 10 and 100 have top surfaces 14, 114 and bottom surfaces 16, 116, respectively, which are substantially parallel to each other such that end surfaces 20, 22 for sleep assist device 10 and 120, 122 for sleep assist device 100 are substantially trapezoidal, as are cross-sections transecting front and back surfaces 12 and 18 for sleep assist device 10 and corresponding surfaces 112 and 118 for sleep assist device 100.

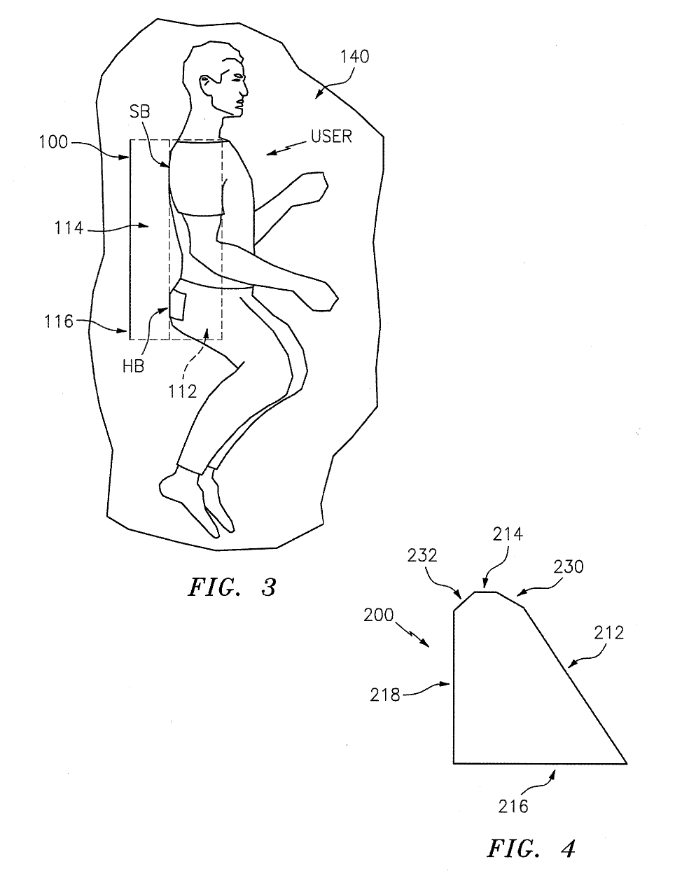

As illustrated schematically in FIG. 3, this invention may also be accomplished by a method to reduce the manifestation of at least one sleep disorder by selecting a sleep assist device such as device 100, FIG. 2. The method further includes placing the bottom surface 116 of the sleep assist device 100 on a bed 140, and placing at least a portion of at least one shoulder blade SB of a user on the sleep assist device. Shoulder blade SB refers to the scapula of the user.

In this example, the user is sleeping on his left side, and at least his right shoulder blade SB rests against the inclined front surface 112. When the left side of the user rests against the bed 140, the left side is sometimes referred to as the “lower side” while the right side of the user is sometimes referred to as the “upper side”. Of course, if the user wants to change sides for sleeping, the device 100 would be rotated 180 degrees while it is lifted above the user and placed on the opposite side of the user. The right side of the user would become the “lower side” while the left side of the user becomes the “upper side”.

Device 100 has a sufficient height H₂, FIG. 2, to support at least the upper portion of the “upper” shoulder blade SB, that is, the shoulder blade furthest away from the bed 140. Device 100 further has sufficient lengths L₂₁ and L₂₂ to support substantially the entire length of shoulder blade SB, and preferably at least some of the ribs of the patient. In the construction shown in FIG. 3, lengths L₂₁ and L₂₂ are sufficient to support the user from the upper portion of shoulder blade SB to a portion of the hip bone HB of the pelvis of the user, also referred to as the sleep assist device having a length that extends substantially along the back of the user. The height and length dimensions can be selected to match anticipated anatomical dimensions of the intended user, such as smaller “pediatric” versions for children and greater dimensions for larger adults.

One suitable source for novel sleep assist devices according to the present invention are based on the “wedge” shape available at www.foamonline.com with inclined front surfaces within the ranges described above. Suitable foams preferably have a density ranging from 0.8 to 4.5 lb and an Indent Load Deflection (“ILD”) value ranging from 10 to 120 lbs, more preferably an ILD from 38 to 50 such as “high density foam” having a foam density of 1.9 lb, “Evlon foam” having a foam density of 2.2 lb, “high resilience foam” having a foam density of 2.8-3.0 lb, or “latex foam” having a foam density of 1.9 lb.

Sleep assist device 200, FIG. 4, has a major front inclined surface 212, a major top surface 214, a bottom surface 216, and a back surface 218. Device 200 further includes minor upper surfaces 230 and 232 which represent alternative “rounding” of the edges where front surface 212 meets top surface 214 and where back surface 218 meets top surface 214, respectively. Device 200 has a corresponding side cross-section that is generally quadrilateral. In yet another construction, top surface 214 is rounded instead of planar.

A currently preferred construction includes a bendable retention tab extending at least six inches from the bottom surface. FIG. 5 illustrates an improved sleep assist device 300 according to the present invention with a bendable, conformable retention tab 310 deployed on a bed Bd. Retention tab 310 extends away from bottom surface 316 by a width W of at least six inches, preferably ten to thirty inches, more preferably twelve to twenty four inches, to assist retention of device 300 against a user as described in more detail below. In this construction, retention tab 310 is formed of a portion of a flexible fabric 311 which covers at least inclined surface 312 with fabric portion 313 and upper surface 314 with fabric portion 315. In other constructions, the fabric serves as a cover or case for the entire device 300. In yet other constructions, fabric or other bendable material forming the retention tab 310 extends from and is secured to at least a portion of bottom surface 316.

FIGS. 6A and 6B are schematic perspective views of the device and bed of FIG. 5 as utilized by a user during sleep. Indentation 330, FIG. 6A, shows how the bendable retention tab conforms to depressions in the bed Bd at “pressure points” such as formed by the arm of the user as illustrated in FIG. 6A. The weight of the user on the retention tab 310 assists retention of the device 300 against the user during sleep, especially resisting sliding of bottom surface 316, FIG. 5, away from the user.

This invention also features manufacturing such a sleep assist device by selecting slightly resilient foam or other suitable pillow-type material, and then forming the material into one of the shapes described above. A preformed block of foam is cut to the desired shape in some constructions and, in other constructions, is formed in a mold having the desired shape.

The slope of the front surface of devices according to the present invention can be made steeper, that is, the lower front angle can be made greater, when more compressible material is selected. In other words, a device made from a foam having greater resilience will compress more than a device formed from a stiffer foam. The “effective angle” is the actual angle achieved when utilized as shown in FIG. 3 above.

Although specific features of the present invention are shown in some drawings and not in others, this is for convenience only, as each feature may be combined with any or all of the other features in accordance with the invention. While there have been shown, described, and pointed out fundamental novel features of the invention as applied to a preferred embodiment thereof, it will be understood that various omissions, substitutions, and changes in the form and details of the devices illustrated, and in their operation, may be made by those skilled in the art without departing from the spirit and scope of the invention. For example, it is expressly intended that all combinations of those elements and/or steps that perform substantially the same function, in substantially the same way, to achieve the same results be within the scope of the invention. Substitutions of elements from one described embodiment to another are also fully intended and contemplated. It is also to be understood that the drawings are not necessarily drawn to scale, but that they are merely conceptual in nature.

It is the intention, therefore, to be limited only as indicated by the scope of the claims appended hereto. Other embodiments will occur to those skilled in the art and are within the following claims.

What is claimed is:

1. A method for reducing the manifestation of at least one sleep disorder for a user, the method comprising:
selecting a sleep assist device which has a generally quadrilateral cross-section with a top surface, a bottom surface, a rear surface, an inclined front surface, and a bendable retention tab extending at least six inches from the bottom surface;
placing the sleep assist device on a bed with the retention tab extending along the bed away from the bottom surface; and
placing at least a portion of at least one shoulder blade of the user on the inclined front surface of the sleep assist device and another portion of the user on the retention tab.

2. The method of claim 1 wherein the front surface is at an effective angle ranging from thirty five degrees to eighty degrees relative to the bottom surface.

3. The method of claim 1 wherein the front surface is at an angle ranging from forty degrees to sixty five degrees relative to the bottom surface.

4. The method of claim 1 wherein the sleep assist device is substantially trapezoidal in cross-section, with the top and bottom surfaces being substantially parallel to each other.

5. The method of claim 4 wherein the front surface is at an angle ranging from forty five degrees to sixty degrees relative to the bottom surface.

6. The method of claim 4 wherein the sleep assist device is selected to be monolithic with the retention tab being formed of a fabric material secured to at least a portion of the monolith.

7. The method of claim 6 wherein the fabric material serves as a removable cover for the monolith.

8. The method of claim 1 wherein the retention tab is a first portion of a fabric material, with another portion of the fabric material covering at least one of the inclined front surface and the bottom surface.

9. The method of claim 1 wherein the sleep assist device has a length that extends substantially along the back of the user and the retention tab extends twelve inches to twenty four inches from the bottom surface.

10. A sleep assist device comprising a generally quadrilateral cross-section with a top surface, a bottom surface, a rear surface, an inclined front surface at an angle ranging from thirty five degrees to eighty degrees relative to the bottom surface, and a bendable retention tab extendable at least six inches along the bed away from the bottom surface.

11. The device of claim 10 wherein the retention tab is a first portion of a fabric material, with another portion of the fabric material covering at least one of the inclined front surface and the bottom surface.

12. The device of claim 11 wherein the front surface is at an angle ranging from forty degrees to sixty five degrees relative to the bottom surface.

13. The device of claim 11 wherein the sleep assist device is substantially trapezoidal in cross-section, with the top and bottom surfaces being substantially parallel to each other.

14. The device of claim 11 wherein the front surface is at an angle ranging from forty five degrees to sixty degrees relative to the bottom surface.

15. The device of claim 10 wherein the sleep assist device is monolithic with the retention tab being formed of a fabric material secured to at least a portion of the monolith.

16. The device of claim 15 wherein the fabric material serves as a removable cover for the monolith.

17. A method for manufacturing a sleep assist device capable of reducing the manifestation of at least one sleep disorder for a user, the method comprising:
selecting a material suitable for a sleep assist device to support at least a portion of a shoulder blade of the user while on a bed; and
forming the sleep assist device to have a generally quadrilateral cross-section with a top surface, a bottom surface, a rear surface, an inclined front surface which is at an angle ranging from thirty degrees to seventy five degrees relative to the bottom surface, and a bendable retention tab extendable along the bed away from the bottom surface.

18. The method of claim 17 wherein the sleep assist device is formed to be monolithic with the retention tab being formed of a fabric material secured to at least a portion of the monolith.

19. The method of claim 18 wherein the fabric material serves as a removable cover for the monolith.
